(19) United States
(12) Patent Application Publication
(10) Pub. No.: US 2023/0295604 A1
(43) Pub. Date: Sep. 21, 2023

(54) Immobilized enzyme, preparation method and use thereof

(71) Applicant: JILIN ASYMCHEM LABORATORIES CO., LTD., Dunhua, Jilin (CN)

(72) Inventors: Hao HONG, Morrisville, NC (US); Gage JAMES, Morrisville, NC (US); Yi XIAO, Tianjin (CN); Na ZHANG, Tianjin (CN); Vyasarayani Williams RAJASEKAR, Tianjin (CN); Yuxia CUI, Tianjin (CN); Jiadong ZHAO, Tianjin (CN); Mingmin HAO, Tianjin (CN); Yanyan GAO, Tianjin (CN)

(21) Appl. No.: 17/789,235

(22) PCT Filed: Dec. 25, 2019
(86) PCT No.: PCT/CN2019/128409

§ 371 Date: Jun. 27, 2022

(51) Int. Cl. C12N 11/091; C12N 9/10; C12N 9/04; C12N 9/02; C12N 9/88; C12N 9/06; C12N 11/06

(52) U.S. Cl. CPC C12N 11/091; C12N 9/0006; C12N 9/0016; C12N 9/0073; C12N 9/88; C12N 9/1096; C12N 11/06; C12Y 104/01009; C12Y 114/13022; C12Y 403/01024

(57) ABSTRACT

Provided is an immobilized enzyme, a preparation method and use thereof. The immobilized enzyme includes an enzyme and an amino resin carrier for immobilizing the enzyme, and the enzyme is selected from any one of the following enzymes: transaminase, ketoreductase, monooxygenase, ammonia-lyase, ene reductase, imine reductase, amino acid dehydrogenase, and nitrilase. The amino resin carrier is an amino resin carrier modified by a cross-linking agent, and the cross-linking agent is a cross-linking agent treated by a polymer. By means of modifying the amino resin carrier with the cross-linking agent treated by the polymer, the enzyme immobilized on the amino resin carrier may easily form a network cross-linking, such that the immobilization effect of the enzyme is more stable, thereby the recycling efficiency of the enzyme is improved.

### CROSS-REFERENCE TO RELATED APPLICATIONS

This application is a national phase application filed under 35 U.S.C. §371 claiming benefit to International Patent Application No. PCT/CN2019/128409, filed on Dec. 25, 2019, the disclosure of which is incorporated by reference herein in its entirety.

### TECHNICAL FIELD

The present invention relates to the field of immobilized enzymes, in particular to an immobilized enzyme, a preparation method and use thereof.

### REFERENCE TO A “SEQUENCE LISTING”, A TABLE, OR A COMPUTER PROGRAM LISTING APPENDIX SUBMITTED AS AN ASCII TEXT FILE

The Sequence Listing written in the ASCII text file: “206418-0013-00US_ReplacementSequenceListing.txt”; created on Nov. 14, 2022, and 62,830 bytes in size, is hereby incorporated by reference.

### BACKGROUND

The applications of microbial cells or separated or engineered enzymes make great progress in biocatalysis, and manufacturing modes are transformed. Many types of enzymes such as an acyltransferase, an amidase, a transaminase, a ketoreductase, an oxidase, a monooxygenase and a hydrolase are used in production of reactions involving antibiotics, herbicides, pharmaceutical intermediates and new age therapeutic agents.

While a free enzyme is used as a biocatalyst, there is a great waste of the enzyme because it is very difficult to recover a water-soluble enzyme. However, a water-insoluble immobilized enzyme may be easily recovered by very simple filtration after each cycle.

Immobilization methods for single enzymes are already reported in existing technologies, but the immobilization methods suitable for the different enzymes are different. For example, Bolivar et al. (Biomacromol. 2006, 7, 669-673) research the covalent immobilization of a formate dehydrogenase (FDH) from pseudomonas SP101, including the covalent immobilization on various carriers such as a modified agarose, a CNBr-activated agarose, Sepabeads (dextran) and an acetaldehyde agarose. It is concluded that the immobilization on activated carriers such as a bromide, a polyethyleneimine, and a glutaraldehyde do not promote any stabilization effects of the enzyme under heat inactivation. However, an optimized enzyme of a highly activated glyoxal agarose is proved to have high heat stability and pH stability,and have more than 50% of the activity in the case of enhanced stability.

Kim et al (J.Mol.Catal B:Enzy 97 (2013) 209-214) report that a method of a cross-linked enzyme aggregate (CLEA) is used to immobilize FDH from Candida boidinii, and it is believed that a dextran polyaldehyde as a cross-linking agent instead of a glutaraldehyde is better for immobilizing the enzyme, and the residual activity exceeds 95% after 10 times of repeated uses. In addition, the heat stability of a cross-linked enzyme aggregate (Dex-CLEA) formed by the dextran polyaldehyde is 3.6 times higher than that of the free enzyme.

Binay et al (Beilstein J. Org. Chem. 2016, 12, 271-277) report a highly active immobilized enzyme of FDH derived from Candida methylica. FDH is covalently immobilized on an epoxy-activated Immobead 150 carrier. The Immobead 150 carrier is firstly modified with an ethylenediamin, then activated with glutaraldehyde (FDHIGLU) and functionalized with an aldehyde group (FDHIALD). The highest immobilization and activity yields are obtained while the aldehyde-functionalized Immobead 150 is used as a carrier, which is 90% and 132%, respectively. At 35° C., the half-lives (t½) of free FDH, FDHI150, FDHIGLU and FDHIALD are respectively calculated to be 10.6, 28.9, 22.4 and 38.5 hours. FDHI150, FDHIGLU and FDHIALD retain 69%, 38%, and 51% of the initial activity thereof respectively after 10 times of repeated uses.

Jackon et al. (Process Biochem. Vol. 1, 9, September 2016, 1248-1255) report immobilization of a lactate dehydrogenase (LDH) using glyoxal-agarose. Compared to a soluble counterpart thereof, a heat stability factor obtained by the immobilized LDH is 1600 times greater.

In conclusion, there are still a large number of enzymes in the existing technologies that do not have effective immobilized enzyme forms, especially some enzymes that jointly participate in the same biocatalytic reaction. The co-immobilization of these enzymes to improve the recyclability of the enzymes becomes an urgent problem to be solved.

### SUMMARY

A main purpose of the present invention is to provide an immobilized enzyme, a preparation method therefor and an application thereof, as to solve a problem in an existing technology that that such enzymes are difficult to recycle.

In order to achieve the above purpose, according to one aspect of the present invention, an immobilized enzyme is provided, and the immobilized enzyme includes an enzyme and an amino resin carrier for immobilizing the enzyme, and the enzyme is selected from any one of the following enzymes: transaminase, ketoreductase, monooxygenase, ammonia-lyase, ene reductase, imine reductase, amino acid dehydrogenase, and nitrilase, the amino resin carrier is an amino resin carrier modified by a cross-linking agent, and the cross-linking agent is a cross-linking agent treated by a polymer.

Further, the transaminase is derived from Chromobacterium violaceum DSM3019, Aspergillus fumigatus, or Arthrobacter citreus, preferably, the transaminase derived from the Chromobacterium violaceum DSM30191 is a mutant with a sequence as shown in SEQ ID NO: 2 or SEQ ID NO: 3; and the transaminase derived from the Arthrobacter citreus is a mutant with a sequence as shown in SEQ ID NO: 5 or SEQ ID NO: 6.

Preferably, the ketoreductase is derived from Acetobacter sp. CCTCC M209061 or Candida macedoniensis AKU4588, more preferably the ketoreductase derived from the Acetobacter sp. CCTCC M209061 is a mutant with a sequence as shown in SEQ ID NO: 8 or SEQ ID NO: 9.

Preferably, the monooxygenase is a cyclohexanone monooxygenase derived from Rhodococcus sp. Phi1, or a cyclohexanone monooxygenase derived from Brachymonas petroleovorans, or a monooxygenase derived from Rhodococcus ruber-SD1, more preferably, the cyclohexanone monooxygenase derived from the Rhodococcus sp. Phi1 is a mutant with a sequence as shown in SEQ ID NO: 11 or SEQ ID NO: 12; and the cyclohexanone monooxygenase derived from the Rhodococcus ruber-SD1 is a mutant with a sequence as shown in SEQ ID NO: 14 or SEQ ID NO: 15.

Preferably, the ammonia lyase is derived from photorhabdus luminescens or Solenostemon scutellarioides; preferably, the ene reductase is derived from Saccharomyces cerevisiae or Chryseobacterium sp. CA49; preferably, the imine reductase is derived from Streptomyces sp or Bacillus cereus; preferably, the amino acid dehydrogenase is a leucine dehydrogenase derived from Bacillus cereus or a phenylalanine dehydrogenase derived from Bacillus sphaericus; and preferably, the nitrilase is derived from Aspergillus niger CBS 513.88 or Neurospora crassa OR74A .

Further, the amino resin carrier is an amino resin carrier with a C2 or C4 linking arm, preferably, the amino resin carrier is selected from any one of the group consisting of: LX1000EA, LX1000HA, LX1000NH, LX1000EPN, HM100D, ECR8309, ECR8409, ECR8305, ECR8404, ECR8315, ECR8415, ESR-1, ESR-3, ESR-5 and ESR-8.

Further the cross-linking agent is a glutaraldehyde, and the macromolecular polymer is a PEG or a PEI; preferably, the PEG is selected from any one of PEG400-PEG6000; preferably, the PEI is selected from a PEI with 3-70 KDa of a molecular weight; more preferably, a mass ratio of the PEG and the glutaraldehyde is 1:1-10:1, further preferably 2:1-5:1; more preferably, a mass ratio of the PEI and the glutaraldehyde is 3:1-1:5, further preferably 1:1-1:2.

In a second aspect of the present application, a preparation method for an immobilized enzyme is provided, and the preparation method includes: performing pretreatment on a cross-linking agent with a macromolecular polymer, to obtain a treated cross-linking agent; performing modification on an amino resin carrier with the treated cross-linking agent, to obtain a modified carrier; and immobilizing an enzyme on the modified carrier, to obtain the immobilized enzyme; herein the enzyme is selected from any one of the group consisting of a transaminase, a ketoreductase, a monooxygenase, an ammonia lyase, an ene reductase, an imine reductase, an amino acid dehydrogenase and a nitrilase.

Further, the transaminase is derived from Chromobacterium violaceum DSM3019, Aspergillus fumigatus, or Arthrobacter citreus, preferably, the transaminase derived from the Chromobacterium violaceum DSM30191 is a mutant with a sequence as shown in SEQ ID NO: 2 or SEQ ID NO: 3; and the transaminase derived from the Arthrobacter citreus is a mutant with a sequence as shown in SEQ ID NO: 5 or SEQ ID NO: 6.

Preferably, the ketoreductase is derived from Acetobacter sp. CCTCC M209061 or Candida macedoniensis AKU4588, more preferably the ketoreductase derived from the Acetobacter sp. CCTCC M209061 is a mutant with a sequence as shown in SEQ ID NO: 8 or SEQ ID NO: 9.

Preferably, the monooxygenase is a cyclohexanone monooxygenase derived from Rhodococcus sp. Phi1, or a cyclohexanone monooxygenase derived from Brachymonas petroleovorans, or a monooxygenase derived from Rhodococcus ruber-SD1, more preferably, the cyclohexanone monooxygenase derived from the Rhodococcus sp. Phi1 is a mutant with a sequence as shown in SEQ ID NO: 11 or SEQ ID NO: 12; and the cyclohexanone monooxygenase derived from the Rhodococcus ruber-SD1 is a mutant with a sequence as shown in SEQ ID NO: 14 or SEQ ID NO: 15.

Preferably, the ammonia lyase is derived from photorhabdus luminescens or Solenostemon scutellarioides; preferably, the ene reductase is derived from Saccharomyces cerevisiae or Chryseobacterium sp. CA49; preferably, the imine reductase is derived from Streptomyces sp or Bacillus cereus; preferably, the amino acid dehydrogenase is a leucine dehydrogenase derived from Bacillus cereus or a phenylalanine dehydrogenase derived from Bacillus sphaericus; and preferably, the nitrilase is derived from Aspergillus niger CBS 513.88 or Neurospora crassa OR74A .

Further, the amino resin carrier is an amino resin carrier with a C2 or C4 linking arm, preferably, the amino resin carrier is selected from any one of the group consisting of: LX1000EA, LX1000HA, LX1000NH, LX1000EPN, HM100D, ECR8309, ECR8409, ECR8305, ECR8404, ECR8315, ECR8415, ESR-1, ESR-3, ESR-5 and ESR-8.

Further, the cross-linking agent is a glutaraldehyde, and the macromolecular polymer is a PEG or a PEI, preferably, the PEG is selected from any one of PEG400-PEG6000; and preferably, the PEI is selected from a PEI with 3-70 KDa of a molecular weight.

Further, a mass ratio of the PEG and the glutaraldehyde is 1:1-10:1, preferably 2:1-5:1; and preferably, a mass ratio of the PEI and the glutaraldehyde is 3:1-1:5, more preferably 1:1-1:2.

According to a third aspect of the present application, an application of any one of the above immobilized enzymes or the immobilized enzyme prepared by any one of the above preparation methods in a biocatalytic reaction is provided.

Further, the biocatalytic reaction is a continuous biocatalytic reaction or a batch reaction, preferably the immobilized enzyme is recycled by 6-16 times under conditions of both aqueous and organic phase reaction.

By applying a technical scheme of the present invention, the amino resin carrier is modified by the cross-linking agent treated with the polymer, it is beneficial to make the enzyme immobilized on it to form a network cross-linking, thereby the immobilization effect of the above enzyme is more stable, and the recycling efficiency of these enzymes is improved.

### DETAILED DESCRIPTION OF THE EMBODIMENTS

It should be noted that embodiments in the present application and features of the embodiments may be combined with each other in the case without conflicting. The present invention is described in detail below with reference to the embodiments.

Amino resin: the resin may be pre-activated with a glutaraldehyde before being used for enzyme immobilization, and then an aldehyde group on the carrier reacts with an amino group on an enzyme molecule to form a Schiff base, to construct a firm multi-site covalent bonding site. It has a long or short amino linking arm.

Enzyme adsorption resin carrier: this type of the resin carrier immobilizes the enzyme on the surface of the water-insoluble carrier resin by a principle of physical adsorption. The immobilization method is mild, hardly changes the conformation of the enzyme, and does not damage an active center of the enzyme. It is especially suitable for the immobilization in an organic solvent or a hydrophobic solvent, and any additional reagents are not required in the immobilization process.

The ion adsorption enzyme carrier resin may form an ionic interaction force with the enzyme molecule within the higher ionic strength, thereby the enzyme is adsorbed and immobilized. The adsorption thereof is reversible, but an adsorption force is stronger than the Van Der Waals force. While the enzyme activity disappears, the carrier may be recycled and reused.

As mentioned in the background, there are still many enzymes that do not achieve the immobilization in existing technologies, and the recycling rate is limited. In order to improve this situation, in a typical embodiment of the present application, an immobilized enzyme is provided, and the immobilized enzyme includes an enzyme and an amino resin carrier for immobilizing the enzyme, and the enzyme is selected from any one of the following enzymes: transaminase, ketoreductase, monooxygenase, ammonia-lyase, ene reductase, imine reductase, amino acid dehydrogenase, and nitrilase, the amino resin carrier is an amino resin carrier modified by a cross-linking agent, and the cross-linking agent is a cross-linking agent treated by a polymer.

The amino resin carrier is modified by the cross-linking agent treated with the polymer, it is beneficial to make the enzyme immobilized on it to form a network cross-linking, thereby the immobilization effect of the above enzyme is more stable, and the recycling efficiency of these enzymes is improved.

In the above immobilized enzymes, the immobilization form of the enzyme on the amino resin carrier is not limited, and it may be covalently immobilized, or non-covalently immobilized. The covalently immobilized form is more stable, so in a preferred embodiment, the enzyme is covalently immobilized on the amino resin carrier.

The specific type of the enzyme in the above immobilized enzymes is selected from any one of transaminase (referred to as TA in the present application), ketoreductase (referred to as KRED in the present application), cyclohexanone monooxygenase (referred to as CHMO in the present application), phenylalanine ammonia lyase (referred to as PLA in the present application), ene reductase (referred to as ERED in the present application), imine reductase (referred to as IRED in the present application), amino acid dehydrogenase (referred to as AADH in the present application) and nitrilase (referred to as NIT in the present application).

The chemical process involved in reactions of the above enzymes is briefly described as follows:

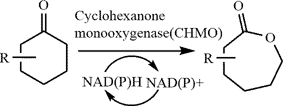

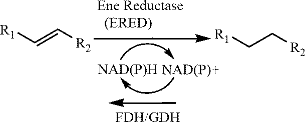

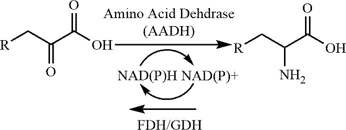

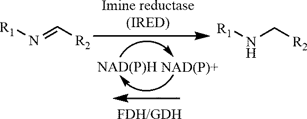

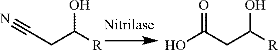

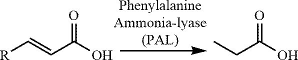

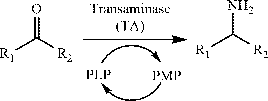

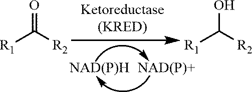

R, R1 and R2 in the above reaction formula may be independently selected from H, a substituted or unsubstituted alkyl, a substituted or unsubstituted cycloalkyl, a substituted or unsubstituted aralkyl, a substituted or unsubstituted heterocyclyl, a substituted or unsubstituted heterocycloalkyl, or R1 and a heterocycle to which it is linked form a fused ring system.

The specific types or species sources of the above enzymes are also not particularly limited, and the types used may be selected according to actual needs. In a preferred embodiment of the present application, the transaminase is derived from Chromobacterium violaceum DSM30191, Aspergillus fumigatus, or Arthrobacter citreus, preferably, the transaminase derived from the Chromobacterium violaceum DSM30191 is a mutant with a sequence as shown in SEQ ID NO: 2 or SEQ ID NO: 3; and the transaminase derived from the Arthrobacter citreus is a mutant with a sequence as shown in SEQ ID NO: 5 or SEQ ID NO: 6.

In another preferred embodiment of the present application, the ketoreductase is derived from Acetobacter sp. CCTCC M209061 or Candida macedoniensis AKU4588, more preferably the ketoreductase derived from the Acetobacter sp. CCTCC M209061 is a mutant with a sequence as shown in SEQ ID NO: 8 or SEQ ID NO: 9.

In another preferred embodiment of the present application, the monooxygenase is a cyclohexanone monooxygenase derived from Rhodococcus sp. Phi1, or a cyclohexanone monooxygenase derived from Brachymonas petroleovorans, or a monooxygenase derived from Rhodococcus ruber-SD1, more preferably, the cyclohexanone monooxygenase derived from the Rhodococcus sp. Phi1 is a mutant with a sequence as shown in SEQ ID NO: 11 or SEQ ID NO: 12; and the cyclohexanone monooxygenase derived from the Rhodococcus ruber-SD1 is a mutant with a sequence as shown in SEQ ID NO: 14 or SEQ ID NO: 15.

In another preferred embodiment of the present application, the ammonia lyase is derived from photorhabdus luminescens or Solenostemon scutellarioides; preferably, the ene reductase is derived from Saccharomyces cerevisiae or Chryseobacterium sp. CA49; preferably, the imine reductase is derived from Streptomyces sp or Bacillus cereus; preferably, the amino acid dehydrogenase is a leucine dehydrogenase derived from Bacillus cereus or a phenylalanine dehydrogenase derived from Bacillus sphaericus; and preferably, the nitrilase is derived from Aspergillus niger CBS 513.88 or Neurospora crassa OR74A .

It should be noted that the above enzymes from various sources, if they are not specifically marked as mutants, are all wild-type, and the specific sequences may be obtained and queried from National Center of Biotechnology Information (NCBI).

The amino resin carrier in the above immobilized enzymes may be a commercially available type. In the present application, preferably the amino resin carrier is an amino resin carrier with a C2 or C4 linking arm. There is also a certain difference in the immobilization effect of the same carrier to the different enzymes. The specific type of the amino resin carrier with the C2 and C4 linking arm may be optimally selected from the existing types according to the different actual enzyme types. In a preferred embodiment of the present application, the amino resin carrier is selected from any one of the group consisting of: LX1000EA, LX1000HA, LX1000NH, LX1000EPN, HM100D, ECR8309, ECR8409, ECR8305, ECR8404, ECR8315, ECR8415, ESR-1, ESR-3, ESR-5 and ESR-8.

Herein, the LX1000EA, LX1000HA, LX1000NH, LX1000EPN, and HM100D are products of SUNRISE Company, the ECR8309, ECR8409, ECR8305, ECR8404, and ECR8315 are products of Purolite Company, and the ESR-1, ESR-3, ESR-5 and ESR-8 are products of Nankai Synthetic Company.

In the above types, the LX1000HA, ECR8409 and LX1000EPN carriers have the relatively best immobilization effects on the transaminase; the LX1000HA and ESR-1 carriers have the relatively best immobilization effects on the ketoreductase; the LX1000HA and ECR8409 carriers have the relatively best immobilization effects on the cyclohexanone monooxygenase; the LX1000HA, LX1000EA, ECR8309 and ECR8409 carriers have the relatively best immobilization effects on the ene reductase; the LX1000HA carrier has the relatively best immobilization effect on the nitrilase; the ECR8409 and LX1000EPN have the relatively best immobilization effects on the imine reductase; the LX1000EPN and ECR8309 have the relatively best immobilization effects on the ammonia lyase; and the LX1000HA and ECR8409 carriers have the relatively best immobilization effects on the amino acid dehydrogenase.

In the above immobilized enzymes, the cross-linking agent is a glutaraldehyde, and the preferred macromolecular polymer is polyethylene glycol (PEG) or polyethylene imine (PEI); preferably, PEG is selected from any one of PEG400~PEG6000; preferably, PEI is selected from PEI with a molecular weight of 3~70 KDa; more preferably, the mass ratio of PEG to the glutaraldehyde is 1:1~10:1, further preferably 2:1~5:1; more preferably, the mass ratio of PEI to the glutaraldehyde is 3:1~1:5, further preferably 1:1~1:2.

In the above preferred embodiment, the macromolecular polymer PEG or PEI is used to treat the glutaraldehyde, so that an aldehyde group of the glutaraldehyde is covalently bonded with a hydroxyl group of PEG or an amino group of PEI, and finally a network structure in which the aldehyde group, and the amino group/hydroxyl group are dispersed is formed, each functional group in this network structure is combined with an enzyme protein through covalent interaction, hydrogen bond interaction, ionic interaction, and hydrophobic interaction and the like, rather than just the covalent interaction like the glutaraldehyde. The covalent bond interaction may easily destroy the activity of the enzyme.

The specific molecular weight of PEG or PEI may be reasonably optimally selected according to the different types of the immobilized enzymes. Within the above molecular weight range, the immobilization effect on the existing enzymes is relatively better. While the mass ratio of the glutaraldehyde treated with PEG or PEI is within the above range, the distribution of the aldehyde group and the amino group/hydroxyl group in a cross-linking agent-polymer composition with the network structure is relatively uniform. If a proportion of the macromolecular polymer is too low, there are more free aldehyde groups, and a spacing between the aldehyde groups is small, a covalent bonding mode dominates the combining with the enzyme protein, so that the enzyme activity is relatively low. If the macromolecular polymer ratio is too high, the amount of the free aldehyde groups is too small, and the covalent bonding becomes weaker while combined with the enzyme protein, and the stability of the immobilized enzyme is decreased. In a preferred range, in the cross-linking agent-polymer composition with the network structure, the ratio and distribution of the aldehyde group to the amino group/hydroxyl group are better, and in the combination with the enzyme, the covalent interaction, the hydrogen bond interaction, the ionic interaction and the hydrophobic interaction and the like are combined in the better ratio, to further improve the activity and stability of the immobilized enzyme. Other macromolecular polymers with hydroxyl functional groups, such as polyvinyl alcohol, may also be used, but the water solubility thereof is poor at a room temperature, and the application effect is limited, so PEG is preferred. Other macromolecular polymers with amino functional groups, such as a polyetheramine, also have a certain effect, but it is considered that it is easy to cause the denaturation of the enzyme protein to a certain extent, PEI is preferred.

In a second typical embodiment of the present application, a preparation method for an immobilized enzyme is provided, and the preparation method includes: performing pretreatment on a cross-linking agent with a macromolecular polymer, to obtain a treated cross-linking agent; performing modification on an amino resin carrier with the treated cross-linking agent, to obtain a modified carrier; and immobilizing an enzyme on the modified carrier, to obtain the immobilized enzyme; herein the enzyme is selected from any one of the group consisting of a transaminase, a ketoreductase, a monooxygenase, an ammonia lyase, an ene reductase, an imine reductase, an amino acid dehydrogenase and a nitrilase.

In the preparation method of the present application, the cross-linking agent has more network structures after being pre-treated with the macromolecular polymer, and then the amino resin carrier is modified by using the cross-linking agent with the more network structures, so that the carrier also has the more network structures. Therefore, while the enzyme is immobilized on the modified carrier, the immobilization effect on the enzyme is more stable.

In another preferred embodiment of the present application, the transaminase is derived from Chromobacterium violaceum DSM30191, Aspergillus fumigatus, or Arthrobacter citreus, preferably, the transaminase derived from the Chromobacterium violaceum DSM30191 is a mutant with a sequence as shown in SEQ ID NO: 2 or SEQ ID NO: 3; and the transaminase derived from the Arthrobacter citreus is a mutant with a sequence as shown in SEQ ID NO: 5 or SEQ ID NO: 6.

In another preferred embodiment of the present application, the ketoreductase is derived from Acetobacter sp. CCTCC M209061 or Candida macedoniensis AKU4588, more preferably the ketoreductase derived from the Acetobacter sp. CCTCC M209061 is a mutant with a sequence as shown in SEQ ID NO: 8 or SEQ ID NO: 9.

In another preferred embodiment of the present application, the monooxygenase is a cyclohexanone monooxygenase derived from Rhodococcus sp. Phi1, or a cyclohexanone monooxygenase derived from Brachymonas petroleovorans, or a monooxygenase derived from Rhodococcus ruber-SD1, more preferably, the cyclohexanone monooxygenase derived from the Rhodococcus sp. Phi1 is a mutant with a sequence as shown in SEQ ID NO: 11 or SEQ ID NO: 12; and the cyclohexanone monooxygenase derived from the Rhodococcus ruber-SD1 is a mutant with a sequence as shown in SEQ ID NO: 14 or SEQ ID NO: 15.

In another preferred embodiment of the present application, the ammonia lyase is derived from photorhabdus luminescens or Solenostemon scutellarioides; preferably, the ene reductase is derived from Saccharomyces cerevisiae or Chryseobacterium sp. CA49; preferably, the imine reductase is derived from Streptomyces sp or Bacillus cereus; preferably, the amino acid dehydrogenase is a leucine dehydrogenase derived from Bacillus cereus or a phenylalanine dehydrogenase derived from Bacillus sphaericus; and preferably, the nitrilase is derived from Aspergillus niger CBS 513.88 or Neurospora crassa OR74A .

The enzyme derived from the above species is immobilized on the amino resin carrier modified by the cross-linking agent treated with the macromolecular polymer, as to form the immobilized enzyme which has the relatively better activity and stability, so the number of cycles is also higher.

In the above preparation method, the amino resin carrier may select a suitable carrier according to the different enzymes. In a preferred embodiment, the amino resin carrier is an amino resin carrier with a C2 or C4 linking arm, preferably, the amino resin carrier is selected from any one of the followings: LX1000EA, LX1000HA, LX1000NH, LX1000EPN, HM100D, ECR8309, ECR8409, ECR8305, ECR8404, ECR8315, ECR8415, ESR-1, ESR-3, ESR-5 and ESR-8. The selection of the above types of case resin carriers is beneficial to the immobilization of the various enzymes.

In the above preparation method, the cross-linking agent may be optimally selected from the existing types of the cross-linking agents according to actual needs. The main function of the macromolecular polymer is to combine the free aldehyde group in the cross-linking agent, thereby a cross-linking agent-polymer composition with the network structure is formed, as to reduce the influence on the activity of the enzyme to be immobilized.

In a preferred embodiment, the cross-linking agent is a glutaraldehyde, and the macromolecular polymer is PEG or PEI. Preferably, PEG is selected from any one of PEG400 ~ PEG6000; and preferably, PEI is selected from PEI with a molecular weight of 3~70 KDa.

In the above preferred embodiment, the specific molecular weight of PEG or PEI may be reasonably optimally selected according to the different types of the immobilized enzymes. Within the above molecular weight range, the immobilization effect on the existing enzymes is relatively better.

In the above preparation method, the mass ratio of PEG to the glutaraldehyde is 1:1~10:1, preferably 2:1~5:1; and preferably, the mass ratio of PEI to the glutaraldehyde is 3:1~1:5, more preferably 1:1~1:2.

The macromolecular polymers with the hydroxyl group or the amino group are all suitable for the present application, but PGE or PEI is preferably used in the above embodiments. Other macromolecular polymers with the hydroxyl functional group, such as polyvinyl alcohol, may also be used, but the water solubility thereof is poor at a room temperature, and the application effect is limited, so PEG is preferred. Other macromolecular polymers with the amino functional group, such as polyetheramine, also have a certain effect, but it is considered that it is easy to cause the denaturation of the enzyme protein to a certain extent, PEI is preferred.

While the mass ratio of the glutaraldehyde treated with PEG or PEI is within the above range, the distribution of the aldehyde group and the amino group/hydroxyl group in the cross-linking agent-polymer composition with the network structure is relatively uniform. If a proportion of the macromolecular polymer is too low, there are more free aldehyde groups, and a spacing between the aldehyde groups is small, a covalent bonding mode dominates the combining with the enzyme protein, so that the enzyme activity is relatively low. If the macromolecular polymer ratio is too high, the amount of the free aldehyde groups is too small, and the covalent bonding becomes weaker while combined with the enzyme protein, and the stability of the immobilized enzyme is decreased. In a preferred range, in the cross-linking agent-polymer composition with the network structure, the ratio and distribution of the aldehyde group to the amino group/hydroxyl group are better, and in the combination with the enzyme, the covalent interaction, the hydrogen bond interaction, the ionic interaction and the hydrophobic interaction and the like are combined in the better ratio, to further improve the activity and stability of the immobilized enzyme.

In the third typical embodiment of the present application, an application of any one of the above immobilized enzymes or the immobilized enzyme prepared by any one of the above preparation methods in a biocatalytic reaction is provided. The immobilized enzyme has the advantages of high stability and high recycling efficiency, so it may be used repeatedly in a biocatalytic reaction.

In a more preferred embodiment, the biocatalytic reaction used by the above immobilized enzyme is a continuous biocatalytic reaction or a batch reaction. The immobilized enzyme has the high recycling efficiency, so it is suitable for the continuous biocatalytic reaction, and improves the reaction efficiency.

The enzyme derived from the above species is immobilized on the amino resin carrier modified by the cross-linking agent treated with the macromolecular polymer, as to form the immobilized enzyme which has the relatively better activity and stability, so the number of cycles is also higher. In a preferred embodiment, the number of cycling times of the above immobilized enzyme under the reaction conditions of an aqueous phase or an organic phase is 6 to 16 times.

The beneficial effects of the present application are further described below with reference to the specific embodiments.

The enzymes used in the following embodiments and the sources thereof are shown in Table 1 below. The sequences of some of the enzymes are shown in Tables 2 to 6.

TABLE 1

| | | |
|---|---|---|
| Short name | Enzyme | Source |
| TA-Af | Transaminase | Aspergillus fumigatus |
| TA-Ac | Transaminase | Arthrobacter citreus |
| TA-Cv | Transaminase | Chromobacterium violaceum DSM30191 |
| KRED-Ac | Ketoreductase | Acetobacter sp. CCTCC M209061 |
| KRED-Cm | Ketoreductase | Candida macedoniensis. AKU4588 |
| CHMO-Bp | Cyclohexanone monooxygenase | Brachymonas petroleovorans |
| CHMO-Rr | Cyclohexanone monooxygenase | Rhodococcus ruber-SDI |
| CHMO-Rs | Cyclohexanone monooxygenase | Rhodococcus sp. Phil |
| ERED-Sc | Ene reductase | Saccharomyces cerevisiae |
| ERED-Chr | Ene reductase | Chryseobacterium sp. CA49 |
| NIT-An | Nitrilase | Aspergillus niger CBS 513.88 |
| NIT-Nc | Nitrilase | Neurospora crassa OR74A |
| IRED-Str | Imine reductase | Streptomyces sp. |
| IRED-Bc | Imine reductase | Bacillus cereus |
| PAL-An | Phenylalanine ammonia lyase | photorhabdus luminescens |
| PAL-Ss | Phenylalanine ammonia lyase | Solenostemon scutellarioides |
| AADH-Bc | Leucine dehydrogenase | Bacillus cereus |
| AADH-Bs | Phenylalanine dehydrogenase | Bacillus sphaericus |

TABLE 2

| | | |
|---|---|---|
| TA-Cv | Sequence number | Sequence |
| Female parent | SEQ ID NO:1 | MQKQRTTSQWRELDAAHHLHPFTDTASLNQAGARVM TRGEGVYLWDSEGNKIIDGMAGLWCVNVGYGRKDFA EAARRQMEELPFYNTFFKTTHPAVVELSSLLAEVTPAG FDRVFYTNSGSESVDTMIRMVRRYWDVQGKPEKKTLI GRWNGYHGSTIGGASLGGMKYMHEQGDLPIPGMAHI EQPWWYKHGKDMTPDEFGWAARWLEEKILEIGADKV AAFVGEPIQGAGGVIVPPATYWPEIERICRKYDVLLVAD EVICGFGRTGEWFGHQHFGFQPDLFTAAKGLSSGYLPI GAVFVGKRVAEGLIAGGDFNHGFTYSGHPVCAAVAHA NVAALRDEGIVQRVKDDIGPYMQKRWRETFSRFEHVD DVRGVGMVQAFTLVKNKAKRELFPDFGEIGTLCRDIFF RNNLIMRACGDHIVSAPPLVMTRAEVDEMLAVAERCLE EFEQTLKARGLA |
| Mutant1 (TA-Cv-V1) | SEQ ID NO:2 | R416T+T7C+S47C+Q380L |
| Mutant 2 (TA-CV-V2) | SEQ ID NO:3 | R416T+T7C+S47C+R405E+K90G+A95P+K304D+Q380 L+I297L |

TABLE 3

| | | |
|---|---|---|
| TA-Ac | Sequence number | Sequence |
| Female parent | SEQ ID NO: 4 | MGLTVQKINWEQVKEWDRKYLMRTFSTQNEYQPVPIES TEGDYLITPGGTRLLDFFNQLCCVNLGQKNQKVNAAIKE ALDRYGFVWDTYATDYKAKAAKIIIEDILGDEDWPGKVRF VSTGSEAVETALNIARLYTNRPLVVTREHDYHGWTGGA ATVTRLRSFRSGLVGENSESFSAQIPGSSCSSAVLMAPS SNTFQDSNGNYLKDENGELLSVKYTRRMIENYGPEQVA AVITEVSQGVGSTMPPYEYVPQIRKMTKELGVLWISDEV LTGFGRTGKWFGYQHYGVQPDIITMGKGLSSSSLPAGA VVVSKEIAAFMDKHRWESVSTYAGHPVAMAAVCANLEV MMEENLVEQAKNSGEYIRSKLELLQEKHKSIGNFDGYGL LWIVDIVNAKTKTPYVKLDRNFRHGMNPNQIPTQIIMEKA LEKGVLIGGAMPNTMRIGASLNVSRGDIDKAMDALDYAL DYLESGEWQQS |
| Mutant 1 (TA-Ac-V1) | SEQ ID NO: 5 | L3S+V5S+C60Y+F164L+A178L+S187A+I180V+L370A+G 411D+S186G+Y384F+I389F+V252I+L404Q+E171D |
| Mutant 2 (TA-Ac-V2) | SEQ ID NO: 6 | L3S+V5S+C60Y+F164L+A178L+S187A+I180V+L370A+G 411D+S186G+Y384F+I389F+V252I+E424Q+M423K |

TABLE 4

| | | |
|---|---|---|
| KRED-Ac | Sequence number | Sequence |
| Female parent | SEQ ID NO: 7 | MARVAGKVAIVSGAANGIGKATAQLLAKEGAKWIGDLK EEDGQKAVAEIKAAGGEAAFVKLNVTDEAAWKAAIGQTL KLYGRLDIAVNNAGINYSGSVESTSLEDWRRVQSINLDG VFLGTQVAIEAMKKSGGGSIVNLSSISGLIGDPMLAAYVA SKGGVRLFTKSAALHCAKSGYKIRVNSVHPGYIWTPMVA GLTKEDAAARQKLVDLHPIGHLGEPNDIAYGILYLASDES KFVTGSELVIDGGYTAQ |
| Mutant 1 (KRED-Ac-V1) | SEQ ID NO: 8 | E144S+A94N+N156V |
| Mutant 2 (KRED-Ac-V2) | SEQ ID NO: 9 | E144S+A94T+N156T |

TABLE 5

| | | |
|---|---|---|
| CHMO-Rs | Sequence number | Sequence |
| Female parent | SEQ ID NO:10 | MTAQISPTVVDAVVIGAGFGGIYAVHKLHNEQGLTVVGFDK ADGPGGTWYWNRYPGALSDTESHLYRFSFDRDLLQDGTWKTT YITQPEILEYLESVVDRFDLRRHFRFGTEVTSAIYLEDENLWEVST DKGEVYRAKYVVNAVGLLSAINFPDLPGLDTFEGETIHTAAWPEG KNLAGKRVGVIGTGSTGQQVITALAPEVEHLTVFVRTPQYSVPVG NRPVTKEQIDAIKADYDGIWDSVKKSAVAFGFEESTLPAMSVSEE ERNRIFQEAWDHGGGFRFMFGTFGDIATDEAANEAAASFIRSKIA EIIEDPETARKLMPTGLYAKRPLCDNGYYEVYNRPNVEAVAIKEN PIREVTAKGVVTEDGVLHELDVLVFATGFDAVDGNYRRIEIRGRN GLHINDHWDGQPTSYLGVTTANFPNWFMVLGPNGPFTNLPPSIE TQVEWISDTVAYAERNEIRAIEPTPEAEEEWTQTCTDIANATLFTR GDSWIFGANVPGKKPSVLFYLGGLGNYRNVLAGVVADSYRGFEL KSAVPVTA |
| Mutant 1 (CHMO-Rs-V1) | SEQ ID NO:11 | F508Y+F435N+L438A+T436S+F280V+S441V |
| Mutant 2 (CHMO-Rs-V2) | SEQ ID NO:12 | F508Y+F435N+L438A+T436S+F280V+S441V+L510V |

TABLE 6

| | | |
|---|---|---|
| CHMO-Rr | Sequence number | Sequence |
| Female parent | SEQ ID NO:13 | MTTSIDREALRRKYAEERDKRIRPDGNDQYIRLDHVDGWSH DPYMPITPREPKLDHVTFAFIGGGFSGLVTAARLRESGVESV RIIDKAGDFGGVWYWNRYPGAMCDTAAMVYMPLLEETGYM PTEKYAHGPEILEHCQRIGKHYDLYDDALFHTEVTDLVWQEH DQRWRISTNRGDHFTAQFVGMGTGPLHVAQLPGIPGIESFR GKSFHTSRWDYDYTGGDALGAPMDKLADKRVAVIGTGATAV QCVPELAKYCRELYVVQRTPSAVDERGNHPIDEKWFAQIAT PGWQKRWLDSFTAIWDGVLTDPSELAIEHEDLVQDGWTALG QRMRAAVGSVPIEQYSPENVQRALEEADDEQMERIRARVDE IVTDPATAAQLKAWFRQMCKRPCFHDDYLPAFNRPNTHLVD TGGKGVERITENGVVVAGVEYEVDCIVYASGFEFLGTGYTDR AGFDPTGRDGVKLSEHWAQGTRTLHGMHTYGFPNLFVLQL MQGAALGSNIPHNFVEAARWAAIVDHVLSTGTSSVETTKEA EQAWVQLLLDHGRPLGNPECTPGYYNNEGKPAELKDRLNV GYPAGSAAFFRMMDHWLAAGSFDGLTFR |
| Mutant 1 (CHMO-Rr-V1) | SEQ ID NO:14 | P190L + Y559M + C249V + C393V + C257A + M45T |
| Mutant 2 (CHMO-Rr-V2) | SEQ ID NO:15 | Y559M + P190L + P504V |

PB in the following embodiments represents a phosphate buffer.

### Embodiment 1: Immobilized Transaminase

1 g of an amino resin is taken, and washed with 20 mM of the phosphate buffer (PB for short, pH 7.0) for later use.

160 µL of a glutaraldehyde (50% of aqueous solution) is added to 4 mL of the PB buffer (20 mM, pH 7.0), and an appropriate ratio of PEG or PEI is added. After being incubated at a room temperature for 30-60 min, the above amino resin is added to the solution, and then it is incubated at 20-25° C. for 1-2 h. After being filtered, the modified amino resin is obtained, and then 4 mL of enzyme solution (20-25 mg/mL protein, including a main enzyme and a corresponding coenzyme thereof) is added to the modified amino resin, and then it is incubated at 20° C. for 16-24 h. Finally, it is washed with 20 mM PB (pH 8.0, and 0.5 M NaCl is contained) for 3 times.

Immobilized transaminase activity and reusability test:

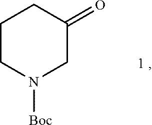 [SMILES: CC(C)(C)OC(=O)N1CCCC(=O)C1]

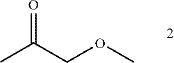

In a 10 mL reaction flask, 0.3 mL of MeOH is added, 0.1 g of a main raw material 1 or a main raw material 2 is added, 4 eq of isopropylamine hydrochloride and 1.0 mg of pyridoxal-5′-phosphate (PLP) are added, and 0.1 M PB 7.0 is supplemented until a final volume of the reaction solution is 1 mL, 0.1 g of enzyme powder or cross-linked enzyme aggregate wet enzyme or cross-linked enzyme aggregate lyophilized powder prepared by 0.1 g of the enzyme powder is added, and it is stirred at 30° C. for 16-20 h. The conversion rate of the system is detected by a High Performance Liquid Chromatography (HPLC), the immobilized enzyme is separated after each round of the reaction, and reused in the next round of the reaction, and the number of repeated uses is investigated. Response data is as follows:

TABLE 7

| Enzyme | Carrier | Mass ratio of macromolecular polymer: cross-linking agent on carrier | Conversion rate (%) | Number of cycles |
|---|---|---|---|---|
| TA-Af | Carrier-free (free enzyme) | -/- | >97% | 1 |
| | LX1000HA | GA | 80%-90% | 5 |
| | | PEG400:GA=10:1 | >97% | 4 |
| | | PEG400:GA=5:1 | >97% | 8 |
| | | PEG400:GA=2:1 | >97% | 8 |
| | | PEG2000:GA=5:1 | >97% | 8 |
| | | PEG2000:GA=2:1 | >97% | 9 |
| | | PEG2000:GA=1:1 | 90% | 9 |
| | | PEG6000:GA=1:2 | 70% | 8 |
| | | PEG6000:GA=2:1 | >97% | 10 |
| | | PEG6000:GA=5:1 | >97% | 9 |
| | | PEG6000:GA=5:2 | >97% | 8 |
| | | PEI (3 KDa):GA=3:1 | >97% | 6 |
| | | PEI (3 KDa):GA=1:1 | >97% | 10 |
| | | PEI (3 KDa):GA=1:5 | 92% | 11 |
| | | PEI (3 KDa):GA=1:7 | 75% | 11 |
| | | PEI (25 KDa):GA=1:1 | >97% | 8 |
| | | PEI (25 KDa):GA=1.5:1 | >97% | 7 |
| | | PEI (25 KDa):GA=1:2 | >97% | 9 |
| | | PEI (70 KDa):GA=1:2 | >97% | 7 |
| | | PEI (70 KDa):GA=1:3 | 90% | 8 |
| | | PEI (70 KDa):GA=1:5 | 85% | 11 |
| | | PEI (70 KDa):GA=1:7 | 75% | 9 |
| | LX1000EPN | GA | 80% | 5 |
| | | PEG400:GA=2:1 | 70%-80% | 3 |
| | | PEG6000:GA=5:2 | 70%-80% | 4 |
| | | PEI (3 KDa):GA=1:1 | 70%-80% | 10 |
| | | PEI (3 KDa):GA=1:2 | 70%-80% | 11 |
| | | PEI (70 KDa):GA=1:5 | 80%-85% | 11 |
| | ECR8409 | GA | 80% | 2 |
| | | PEG6000:GA=5:2 | 70%-80% | 8 |
| | | PEI (70 KDa):GA=1:5 | 80%-85% | 8 |
| | ESR-1 | GA | <70% | 2 |
| | | PEG6000:GA=5:2 | 70%-80% | 4 |
| | ESR-3 | GA | <70% | 2 |
| | | PEG6000:GA=5:2 | 70%-80% | 3 |
| TA-Ac | Free enzyme | -/ | >97% | 1 |
| | LX1000EA | GA | 80%-85% | 4 |
| | | PEG6000:GA=5:2 | 80%-90% | 7 |
| | LX1000HA | GA | >97% | 6 |
| | | PEG6000:GA=5:2 | >97% | 12 |
| | | PEI (3 KDa):GA=1:1 | >97% | 10 |
| | ESR-1 | GA | 80%-90% | 4 |
| | | PEG6000:GA=5:2 | 90%-95% | 6 |
| | | PEI (3 KDa):GA=1:1 | 90%-95% | 6 |
| | ESR-8 | GA | 80%-85% | 3 |
| | | PEG6000:GA=5:2 | 90%-95% | 5 |
| | LX1000EPN | PEG6000:GA=5:2 | >97% | 10 |
| TA-Ac-V1 | LX1000HA | PEG6000:GA=5:2 | >97% | 16 |
| TA-Ac-V2 | LX1000HA | PEG6000:GA=5:2 | >97% | 19 |
| TA-Cv | Free enzyme | -/ | >97% | 1 |
| | ECR8409 | GA | >97% | 6 |
| | | PEG6000:GA=5:2 | >97% | 8 |
| | | PEI (3 KDa):GA=1:1 | >97% | 8 |
| | ECR8404 | PEI (70 KDa):GA=1:5 | >97% | 7 |
| | | GA | >97% | 4 |
| | | PEG6000:GA=5:2 | >97% | 7 |
| | | PEI (3 KDa):GA=1:1 | >97% | 7 |
| | ECR8315 | GA | >97% | 4 |
| | | PEG6000:GA=5:2 | >97% | 8 |
| | | PEI (3 KDa):GA=1:1 | >97% | 7 |
| | LX1000HA | GA | >97% | 6 |
| | | PEG6000:GA=5:2 | >97% | 7 |
| | | PEI (3 KDa):GA=1:1 | >97% | 9 |
| | | PEI (25 KDa):GA=1:5 | >97% | 10 |
| | LX1000EPN | GA | >97% | 6 |
| | | PEG6000:GA=5:2 | >97% | 7 |
| | | PEI (3 KDa):GA=1:1 | >97% | 7 |
| | ESR-01 | GA | >97% | 4 |
| | | PEG6000:GA=5:2 | >97% | 6 |
| | | PEI (3 KDa):GA=1:1 | >97% | 7 |
| | ESR-03 | GA | >97% | 4 |
| | | PEG6000:GA=5:2 | >97% | 5 |
| | | PEI (3 KDa):GA=1:1 | >97% | 5 |
| TA-Cv-V1 | LX1000HA | PEI (25 KDa):GA=1:5 | >97% | 16 |
| | | PEG6000:GA=5:2 | >97% | 19 |
| TA-Cv-V2 | LX1000HA | PEG6000:GA=5:2 | >97% | 22 |

### Embodiment 2: Conversion Rate and Reusability Test of Immobilized Ketoreductase

An immobilization method is the same as in Embodiment 1.

Immobilized enzyme activity and reusability detection:

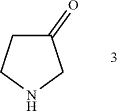

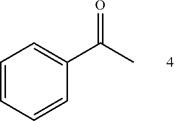

In a 10 mL reaction flask, 0.5 mL of isopropanol (IPA) is added, 0.1 g of a main raw material 3 or 4 is dissolved, 0.5 mL of 0.1 M PB 7.0 and 1-10 mg of nicotinamide adenine dinucleotide (NAD+) are added, then 0.05 g of enzyme powder or the immobilized enzyme prepared by 0.1-0.3 g of the enzyme powder is added. It is stirred at 30° C. for 16-20 h. The conversion rate of the system is detected by a Gas Chromatography (GC), the immobilized enzyme is separated after each round of the reaction, and reused in the next round of the reaction, and the number of repeated uses is investigated. Response data is as follows:

TABLE 8

| Enzyme | Carrier | Mass ratio of macromolecular polymer: cross-linking agent on carrier | Conversion rate (%) | Number of cycles |
|---|---|---|---|---|
| KRED-Ac | Carrier-free (free enzyme) | -/ | >99% | 1 |
| | LX1000HA | GA | >99% | 5 |
| | | PEG6000:GA=5:2 | >99% | 9 |
| | | PEI (3 KDa):GA=1:1 | >99% | 10 |
| | | PEI (3 KDa):GA=1:2 | >99% | 10 |
| | | PEI (70 KDa):GA=1:5 | >99% | 10 |
| | LX1000EA | GA | >99% | 2 |
| | | PEG6000:GA=5:2 | >99% | 5 |
| | | PEI (3 KDa):GA=1:1 | >99% | 6 |
| | LX1000EPN | GA | >99% | 4 |
| | | PEG6000:GA=5:2 | >99% | 7 |
| | | PEI (3 KDa):GA=1:2 | >99% | 7 |
| | ESR-1 | GA | >99% | 3 |
| | | PEI (3 KDa):GA=1:1 | >99% | 5 |
| | ESR-3 | GA | >99% | 2 |
| | | PEI (3 KDa):GA=1:1 | >99% | 5 |
| | ESR-8 | GA | >99% | 2 |
| | | PEI (3 KDa):GA=1:1 | >99% | 4 |
| KRED-Ac-V1 | LX1000EPN | PEG6000:GA=5:2 | >99% | 12 |
| | | PEI (3 KDa):GA=1:1 | >99% | 15 |
| KRED-Ac-V2 | LX1000EPN | PEG6000:GA=5:2 | >99% | 15 |
| KRED-Cm | Free enzyme | -/- | 90% | 1 |
| | LX1000EA | GA | 50%-55% | 4 |
| | | PEG6000:GA=5:2 | 80% | 3 |
| | LX1000HA | GA | 75% | 3 |
| | | PEG6000:GA=5:2 | 75% | 6 |
| | | PEI (3 KDa):GA=1:1 | 75% | 5 |
| | ESR-1 | GA | 50% | 4 |
| | | PEG6000:GA=5:2 | 70% | 6 |
| | | PEI (3 KDa):GA=1:1 | 70% | 6 |
| | ESR-8 | GA | 75% | 3 |
| | | PEG6000:GA=5:2 | 75% | 6 |

### Embodiment 3: Conversion Rate and Reusability Test of Immobilized CHMOs

An immobilization method is the same as in Embodiment 1.

The activity of the CHMO amino carrier immobilized enzyme is detected by reacting with the following substrate 5

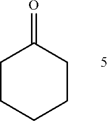

0.3 mL of isopropanol is loaded in a 10 ml reaction flask, and then 500 mg of the substrate 5, and 3 mL of 0.1 M PB (pH 8.0) containing 5 mg of nicotinamide adenine dinucleotide phosphate (NADP+) are added, then 50 mg of alcohol dehydrogenase ADH-Tb free enzyme and 100-200 mg of CHMO amino carrier co-immobilized enzyme (wet, containing 50-80% of water) are added. It is reacted at 30° C. for 16-20 hours, and the conversion rate is tested. The immobilized enzyme is separated after each round of the reaction, and reused in the next round of the reaction, and the number of repeated uses is investigated. Results are shown in the table below.

TABLE 9

| Enzyme | Carrier | Mass ratio of macromolecular polymer: cross-linking agent on carrier | Conversion rate (%) | Number of cycles |
|---|---|---|---|---|
| CHMO-Bp | Carrier-free (free enzyme) | -/ | >98% | 1 |
| | LX1000HA | GA | 80% | 5 |
| | | PEG400:GA=2:1 | 95% | 7 |
| | | PEG2000:GA=2:1 | 95% | 6 |
| | | PEG6000:GA=2:1 | 95% | 6 |
| | | PEG6000:GA=5:2 | 95% | 7 |
| | | PEI (3 KDa):GA=1:1 | 95% | 6 |
| | | PEI (25 KDa):GA=1:2 | 95% | 8 |
| | | PEI (70 KDa):GA=1:5 | 95% | 8 |
| | LX1000EPN | GA | 80% | 4 |
| | | PEG400:GA=2:1 | 95% | 7 |
| | | PEG6000:GA=5:2 | 95% | 6 |
| | | PEI (3 KDa):GA=I:I | 95% | 5 |
| | | PEI (3 KDa):GA=I:2 | 95% | 7 |
| | | PEI (70 KDa):GA=1:5 | 95% | 8 |
| | ECR8415 | GA | 80% | 4 |
| | | PEG6000:GA=5:2 | 95% | 7 |
| | | PEI (70 KDa):GA=1:5 | 95% | 6 |
| | LX1000NH | GA | 80% | 4 |
| | | PEG6000:GA=5:2 | 95% | 6 |
| | ESR-8 | GA | 80% | 5 |
| | | PEG6000:GA=5:2 | 95% | 7 |
| CHMO-Rr | Free enzyme | -/ | >98% | 1 |
| | LX1000EA | GA | 90% | 5 |
| | | PEG6000:GA=5:2 | 98% | 8 |
| | LX1000HA | GA | 90% | 6 |
| | | PEG6000:GA=5:2 | 98% | 9 |
| | | PEI (3 KDa):GA=1:1 | 98% | 8 |
| | ECR8409 | GA | 90% | 6 |
| | | PEG6000:GA=5:2 | 98% | 8 |
| | | PEI (3 KDa):GA=1:1 | 98% | 8 |
| | ESR-8 | GA | 90% | 5 |
| | | PEG6000:GA=5:2 | 98% | 7 |
| CHMO-Rr-V1 | ECR8409 | PEG6000:GA=5:2 | 98% | 11 |
| CHMO-Rr-V2 | ECR8409 | PEG6000:GA=5:2 | 98% | 14 |
| CHMO-Rs | Free enzyme | -/ | >97% | 1 |
| | ECR8409 | GA | 90% | 7 |
| | | PEG6000:GA=5:2 | 98% | 9 |
| | | PEI (3 KDa):GA=1:1 | 98% | 9 |
| | ECR8315 | GA | 90% | 4 |
| | | PEG6000:GA=5:2 | 98% | 8 |
| | | PEI (3 KDa):GA=1:1 | 98% | 7 |
| | LX1000HA | GA | 90% | 6 |
| | | PEG6000:GA=5:2 | 98% | 8 |
| | | PEI (3 KDa):GA=1:1 | 98% | 8 |
| | | PEI (25 KDa):GA=1:5 | 98% | 9 |
| | LX1000EPN | GA | 90% | 5 |
| | | PEG6000:GA=5:2 | 98% | 7 |
| | | PEI (3 KDa):GA=1:1 | 98% | 7 |
| | HM100D | GA | 90% | 4 |
| | | PEG6000:GA=5:2 | 98% | 5 |
| | | PEI (3 KDa):GA=1:1 | 98% | 5 |
| CHMO-Rs-V1 | LX1000EPN | PEI (25 KDa):GA=1:5 | 98% | 10 |
| CHMO-Rs-V2 | LX1000EPN | PEI (25 KDa):GA=1:5 | 98% | 14 |

### Embodiment 4: Conversion Rate and Reusability Test of Immobilized EREDs

An immobilization method is the same as in Embodiment 1.

The activity of the ERED amino carrier immobilized enzyme is detected by reacting with the following substrate 6:

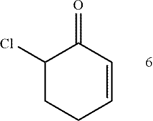

3 mL of 0.1 M PB (pH 7.0-8.0) is loaded in a 10 ml reaction flask, and then 100 mg of the substrate 6 is added, and 10 mg of nicotinamide adenine dinucleotide phosphate (NAD (P)+), 80 mg of an ammonium formate, 20 mg of FDH and 100 mg of ERED immobilized enzyme are added. It is reacted at 30° C. for 16-20 hours, and the conversion rate is tested. The immobilized enzyme is separated after each round of the reaction, and reused in the next round of the reaction, and the number of repeated uses is investigated. Test results are shown in the table below.

TABLE 10

| Enzyme | Carrier | Mass ratio of macromolecular polymer: cross-linking agent on carrier | Conversion rate (%) | Number of cycles |
|---|---|---|---|---|
| ERED-Sc | Carrier-free (free enzyme) | -/ | >90% | 1 |
| | LX1000HA | GA | >90% | 6 |
| | | PEG6000:GA=5:2 | >90% | 8 |
| | | PEI (3 KDa):GA=1:1 | >90% | 8 |
| | | PEI (3 KDa):GA=1:2 | >90% | 9 |
| | | PEI (70 KDa):GA=1:5 | >90% | 10 |
| | LX1000EPN | GA | >90% | 6 |
| | | PEG6000:GA=5:2 | >90% | 9 |
| | | PEI (3 KDa):GA=1:2 | >90% | 9 |
| | ECR8409 | GA | >90% | 7 |
| | | PEI (3 KDa):GA=1:2 | >90% | 10 |
| | ESR-8 | GA | >90% | 5 |
| | | PEI (70 KDa):GA=1:5 | >90% | 7 |
| ERED-Chr | Free enzyme | -/ | >90% | 1 |
| | LX1000EA | GA | >90% | 6 |
| | | PEG6000:GA=5:2 | >90% | 8 |
| | LX1000HA | GA | >90% | 5 |
| | | PEG6000:GA=5:2 | >90% | 8 |
| | | PEI (3 KDa):GA=1:2 | >90% | 8 |
| | ECR8309 | GA | >90% | 6 |
| | | PEG6000:GA=5:2 | >90% | 8 |
| | | PEI (3 KDa):GA=1:1 | >90% | 8 |
| | ESR-3 | GA | >90% | 5 |
| | | PEG6000:GA=5:2 | >90% | 7 |

### Embodiment 5: Conversion Rate and Reusability Test of Immobilized NITs

An immobilization method is the same as in Embodiment 1.

The activity of the NIT amino carrier immobilized enzyme is detected by reacting with the following substrate 7

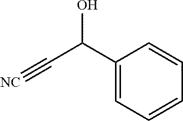

2 mL of 0.1 M PB buffer (pH 7.0-8.0) is added to a 10 mL reaction flask, and 100 mg of the above substrate 9 is added, then the amino carrier immobilized enzyme containing 200 mg of NIT is added. After it is reacted at 30° C. for 16 hours, the conversion rate is detected. The immobilized enzyme is separated after each round of the reaction, and reused in the next round of the reaction, and the number of repeated uses is investigated. Test results are shown in the table below.

TABLE 11

| Enzyme | Carrier | Mass ratio of macromolecular polymer: cross-linking agent on carrier | Conversion rate (%) | Number of cycles |
|---|---|---|---|---|
| NIT-An | Carrier-free (free enzyme) | –/– | >98% | 1 |
| | LX1000HA | GA | >98% | 3 |
| | | PEG6000:GA=5:2 | >98% | 6 |
| | | PEI (3 KDa):GA=1:1 | >98% | 5 |
| | | PEI (3 KDa):GA=1:2 | >98% | 6 |
| | | PEI (70 KDa):GA=1:5 | >98% | 6 |
| | LX1000EPN | GA | >98% | 3 |
| | | PEG6000:GA=5:2 | >98% | 5 |
| | | PEI (3 KDa):GA=1:2 | >98% | 5 |
| | ECR8409 | GA | >98% | 3 |
| | | PEI (3 KDa):GA=1:2 | >98% | 5 |
| NIT-Nc | Free enzyme | –/– | >98% | 1 |
| | LX1000HA | GA | >98% | 3 |
| | | PEG6000:GA=5:2 | >98% | 6 |
| | | PEI (3 KDa):GA=1:1 | >98% | 5 |
| | ECR8409 | GA | >98% | 4 |
| | | PEG6000:GA=5:2 | >98% | 6 |
| | | PEI (3 KDa):GA=1:1 | >98% | 6 |
| | ESR-1 | GA | >98% | 3 |
| | | PEG6000:GA=5:2 | >98% | 6 |

### Embodiment 6: Conversion Rate and Reusability Test of Immobilized IREDs

An immobilization method is the same as in Embodiment 1.

The activity of the IRED amino carrier immobilized enzyme is detected by reacting with the following substrate 8

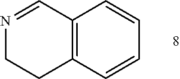

2 mL of 0.1 M PB buffer (pH 7.0-8.0) is added to a 10 mL reaction flask, and then 100 mg of the above substrate 8, 10 mg of NAD+, 60 mg of an ammonium formate, 10 mg of FDH and the immobilized enzyme containing 400 mg of the IRED amino carrier is added. After it is reacted at 30° C. for 20 hours, the conversion rate is detected. The immobilized enzyme is separated after each round of the reaction, and reused in the next round of the reaction, and the number of repeated uses is investigated. Test results are shown in the table below.

TABLE 12

| Enzyme | Carrier | Mass ratio of macromolecular polymer: cross-linking agent on carrier | Conversion rate (%) | Number of cycles |
|---|---|---|---|---|
| IRED-Str | Carrier-free (free enzyme) | –/– | >98% | 1 |
| | LX1000HA | GA | >90% | 2 |
| | | PEG6000:GA=5:2 | >90% | 3 |
| | | PEI (70 KDa):GA=1:5 | >90% | 3 |
| | LX1000EPN | GA | >90% | 2 |
| | | PEG6000:GA=5:2 | >90% | 3 |
| | | PEI (3 KDa):GA=1:2 | >90% | 3 |
| | ECR8409 | GA | >90% | 2 |
| | | PEI (3 KDa):GA=1:1 | >90% | 4 |
| | ESR-1 | GA | >90% | 2 |
| | | PEI (3 KDa):GA=1:1 | >90% | 5 |
| IRED-Bc | Free enzyme | –/– | >90% | 1 |
| | LX1000EPN | GA | >90% | 3 |
| | | PEG6000:GA=5:2 | >90% | 5 |
| | LX1000HA | GA | >90% | 3 |
| | | PEG6000:GA=5:2 | >90% | 5 |
| | | PEI (3 KDa):GA=1:2 | >90% | 5 |
| | ECR8309 | GA | >90% | 3 |
| | | PEG6000:GA=5:2 | >90% | 4 |
| | | PEI (3 KDa):GA=1:1 | >90% | 4 |
| | ECR8409 | GA | >90% | 3 |
| | | PEG6000:GA=5:2 | >90% | 5 |

### Embodiment 7: Conversion Rate and Reusability Test of Immobilized PALs

An immobilization method is the same as in Embodiment 1.

The activity of the PAL amino carrier immobilized enzyme is detected by reacting with the following substrate 9

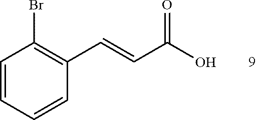 [SMILES: O=C(O)/C=C/c1ccccc1Br]

8 mL of 4 M ammonium carbamate aqueous solution (pH 9.0-9.5) is added to a 10 mL reaction flask, 100 mg of the above substrate 10 is added, and then the immobilized enzyme containing 400 mg of NIT is added. After it is reacted at 30° C. for 16-20 hours, the conversion rate is detected. The immobilized enzyme is separated after each round of the reaction, and reused in the next round of the reaction, and the number of repeated uses is investigated. Test results are shown in the table below.

TABLE 13

| Enzyme | Carrier | Mass ratio of macromolecular polymer: cross-linking agent on carrier | Conversion rate (%) | Number of cycles |
|---|---|---|---|---|
| PAL-An | Carrier-free (free enzyme) | -/- | 80% | 1 |
| | LX1000HA | GA | 80% | 4 |
| | | PEG6000:GA=5:2 | 80% | 5 |
| | | PEI (70 KDa):GA=1:5 | 80% | 6 |
| | LX1000EA | GA | 80% | 4 |
| | | PEG6000:GA=5:2 | 80% | 5 |
| | | PEI (3 KDa):GA=1:1 | 80% | 5 |
| | LX1000EPN | GA | 80% | 6 |
| | | PEG6000:GA=5:2 | 80% | 8 |
| | | PEI (3 KDa):GA=1:2 | 80% | 8 |
| | ECR8309 | GA | 80% | 6 |
| | | PEI (3 KDa):GA=1:1 | 80% | 7 |
| | ECR8315 | GA | 80% | 6 |
| | | PEI (3 KDa):GA=1:1 | 80% | 8 |
| | ESR-8 | GA | 80% | 5 |
| | | PEI (3 KDa):GA=1:1 | 80% | 7 |
| PAL-Ss | Free enzyme | -/- | 80% | 1 |
| | LX1000EA | GA | 80% | 6 |
| | | PEG6000:GA=5:2 | 80% | 9 |
| | LX1000HA | GA | 80% | 6 |
| | | PEG6000:GA=5:2 | 80% | 8 |
| | | PEI (3 KDa):GA=1:1 | 80% | 8 |
| | LX1000EPN | GA | 80% | 9 |
| | | PEG6000:GA=5:2 | 80% | 12 |
| | | PEI (3 KDa):GA=1:1 | 80% | 12 |
| | ECR8309 | GA | 80% | 9 |
| | | PEG6000:GA=5:2 | 80% | 11 |

### Embodiment 8: Conversion Rate and Reusability Test of Immobilized AADHs

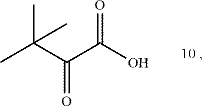

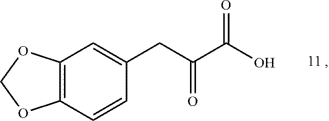 [SMILES: O=C(O)C(=O)Cc1ccc2c(c1)OCO2]

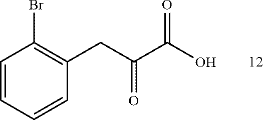 [SMILES: O=C(O)C(=O)Cc1ccccc1Br]

In a 10 mL reaction flask, 5 mL of 0.1 M Tris-CI (pH 8.0-9.0) is added, then 100 mg of the main raw material 10, 11 or 12, and 108 mg of an ammonium chloride are added, a pH is adjusted to 7.5-8.0, then 10-50 mg of NAD+, 50 mg of GDH, and 100 mg of an AADH enzyme (or immobilized AADH prepared by 100-300 mg of a free enzyme) are added. After it is reacted at 30° C. for 16-20 h, it is used for a conversion rate test. Test results are shown in the table below.

TABLE 14

| Enzyme | Carrier | Mass ratio of macromolecular polymer: cross-linking agent on carrier | Conversion rate (%) | Number of cycles |
|---|---|---|---|---|
| AADH-Bc | Carrier-free (free enzyme) | –/– | >99% | 1 |
| | LX1000HA | GA | >99% | 8 |
| | | PEG6000:GA=5:2 | >99% | 12 |
| | | PEI (3 KDa):GA=1:1 | >99% | 12 |
| | LX1000EA | GA | >99% | 5 |
| | | PEG6000:GA=5:2 | >99% | 7 |
| | ECR8409 | GA | >99% | 10 |
| | | PEG6000:GA=5:2 | >99% | 14 |
| | ESR-1 | GA | >99% | 7 |
| | | PEG6000:GA=5:2 | >99% | 10 |
| | ESR-3 | GA | >99% | 7 |
| | | PEG6000:GA=5:2 | >99% | 9 |
| AADH-Bs | Free enzyme | –/– | >99% | 1 |
| | LX1000EA | GA | >99% | 6 |
| | | PEG6000:GA=5:2 | >99% | 9 |
| | LX1000HA | GA | >99% | 9 |
| | | PEG6000:GA=5:2 | >99% | 12 |
| | | PEI (3 KDa):GA=1:1 | >99% | 11 |
| | ESR-1 | GA | >99% | 8 |
| | | PEG6000:GA=5:2 | >99% | 12 |
| | | PEI (3 KDa):GA=1:1 | >99% | 11 |
| | ESR-8 | GA | >99% | 6 |
| | | PEG6000:GA=5:2 | >99% | 8 |

### Embodiment 9: Application of Transaminase Amino Carrier Immobilized Enzyme in Packed Bed continuous reaction

In Embodiment 1, a transaminase TA-Cv is immobilized on a carrier LX1000HA, a cross-linking agent GA is modified by PEI (3 KDa):GA=1:1, and the obtained immobilized enzyme is filled into a column-shaped reactor with a column volume of 120 mL, and the amount of the immobilized enzyme used is 72 g.

500 g of the substrate 1 is dissolved with 1.5 L of methanol, and 4 eq of an isopropylamine hydrochloride (1.8 L of 6 M isopropylamine hydrochloride in water) and 5 g of PLP are added without adding a PB buffer (0.1 M, pH 8.0), and the volume is fixed to 5 L.

The flow rate is set to 0.6 mL/min, namely the retention time is 200 min, a continuous reaction is performed, effluent liquid at an outlet is tested for the conversion rate, and the conversion rate is > 98%. After 400 h of continuous operation, the conversion rate is not decreased. After 420 h of the operation, the conversion rate is decreased to 89%. See the table below for details.

TABLE 15

| Enzyme | Carrier | Immobilized enzyme amount | Column volume | Retention time | Operation time | Conversion rate |
|---|---|---|---|---|---|---|
| TA-Cv-V1 | LX1000HA | 72 g | 120 mL | 200 min | 400 h | 97.5% |
| | | | | | 420 h | 89% |

### Embodiment 10: Application of Transaminase Immobilized Enzyme in Continuous Stirred Tank reaction

The same immobilized enzyme TA-Ac in Embodiment 1 is used, the carrier is ECR8409, and the used cross-linking agent GA is modified with PEG6000:GA=5:2. 50 g of the immobilized enzyme of transaminase TA-Ac is added to a 200 mL reactor, and 150 mL of a phosphate buffer is added.

500 g of the substrate 1 is added with 3.2 L of PB (0.1 M, pH 7.0), 1.8 L of isopropylamine hydrochloride aqueous solution (6 M) and 5 g of PLP, and it is beaten to prepare suspension.

The substrate suspension is continuously added to a reaction flask at a rate of 0.4 mL/min (namely the retention time is 500 min), and a reaction system is extracted from an outlet at the same flow rate (a filter heat is added at a tail end of a pipe to prevent the immobilized enzyme from being extracted). Under this condition, the conversion rate may reach more than 90%, and the conversion rate is basically not reduced after continuous operation for 400 h. Results are shown in the table below.

TABLE 16

| Enzyme | Carrier | Immobilized enzyme amount | Reactor volume | Retention time | Operation time | Conversion rate |
|---|---|---|---|---|---|---|
| TA-Cv | ECR8409 | 50 g | 200 mL | 250 min | 400 h | >90% |
| | | | | | 424 h | 82% |

### Embodiment 11

According to the ammonia lyase PAL-Ss immobilized enzyme prepared in Embodiment 7, the carrier is LX1000EPN, and the cross-linking agent is modified with PEG6000:GA=5:2. 6 g of the obtained immobilized enzyme is filled into a 10 mL column-shaped reactor.

500 g of the substrate 9 is dissolved with 4.5 L of ammonium carbamate aqueous solution (4 M, pH 9.0-9.5).

The flow rate is set to 0.03 mL/min, namely the retention time is 330 min, and a continuous reaction is performed. Effluent liquid at an outlet is tested for the conversion rate, and the conversion rate is 80%. After 360 h of continuous operation, the conversion rate is not decreased, and after 400 h of the operation, the conversion rate is decreased to 72%. See the table below.

TABLE 17

| Enzyme | Carrier | Immobilized enzyme amount | Column volume | Retention time | Operation time | Conversion rate |
|---|---|---|---|---|---|---|
| PAL-Ss | LX1000EPN | 6 g | 10 mL | 100 min | 360 h | 80% |
| | | | | | 400 h | 72% |

### Embodiment 12

According to the ketoreductase KRED-Ac immobilized enzyme prepared in Embodiment 2, the carrier is LX1000HA, and the cross-linking agent is modified by PEI (3 KDa):GA=1:2. 6 g of the obtained immobilized enzyme is filled into a 10 mL column-shaped reactor.

100 g of the substrate 3 is dissolved with 0.3 L of isopropanol, and 0.7 L of PB buffer (0.1 M, pH 7.0) is added to dissolve, and then 0.1 g of NAD + is added.

The flow rate is set to 0.05 mL/min, namely the retention time is 200 min, and a continuous reaction is performed. Effluent liquid at an outlet end is detected for the conversion rate, and the conversion rate is >90%. The conversion rate is not reduced after 200 h of continuous operation, and the conversion rate is reduced to 84% after 220 h of the operation. See the table below.

TABLE 18

Reaction result of KRED-Ac immobilized enzyme in packed bed continuous reaction

| Enzyme | carrier | Immobilized enzyme amount | Column volume | Retention time | Operation time | Conversion rate |
|---|---|---|---|---|---|---|
| KRED-AC+GDH | LX1000HA | 5.9 g | 10 mL | 100 min | 200 h | 90% |
| | | | | | 220 h | 84% |

From the above description, it may be seen that the above embodiments of the present invention achieve the following technical effects: the amino resin carrier is modified by using the cross-linking agent treated with the macromolecular polymer, it is beneficial to make the enzyme immobilized on it to form a network cross-linking, thereby the immobilization effect of the enzyme is more stable, and the recycling efficiency of the enzyme is improved.

The above are only preferred embodiments of the present invention and are not intended to limit the present invention. For those skilled in the art, the present invention may have various modifications and changes. Any modifications, equivalent replacements, improvements and the like made within the spirit and principle of the present invention shall be included within a scope of protection of the present invention.

What is claimed is:

1. An immobilized enzyme, comprising an enzyme and the enzyme is immobilized on an amino resin carrier, wherein
the enzyme is selected from any one of the group consisting of a transaminase, a ketoreductase, a monooxygenase, an ammonia lyase, an ene reductase, an imine reductase, an amino acid dehydrogenase and a nitrilase, and
the amino resin carrier is modified by a cross-linking agent, and the cross-linking agent is a macromolecular polymer modified cross-linking agent.

2. The immobilized enzyme as claimed in claim 1, wherein the transaminase is derived from Chromobacterium violaceum DSM3019, Aspergillus fumigatus, or Arthrobacter citreus, preferably, the transaminase derived from the Chromobacterium violaceum DSM30191 is a mutant with a sequence as shown in SEQ ID NO: 2 or SEQ ID NO: 3; and the transaminase derived from the Arthrobacter citreus is a mutant with a sequence as shown in SEQ ID NO: 5 or SEQ ID NO: 6;
preferably, the ketoreductase is derived from Acetobacter sp. CCTCC M209061 or Candida macedoniensis AKU4588, more preferably the ketoreductase derived from the Acetobacter sp. CCTCC M209061 is a mutant with a sequence as shown in SEQ ID NO: 8 or SEQ ID NO: 9;
preferably, the monooxygenase is a cyclohexanone monooxygenase derived from Rhodococcus sp. Phil, or a cyclohexanone monooxygenase derived from Brachymonas petroleovorans, or a monooxygenase derived from Rhodococcus ruber-SD1, more preferably, the cyclohexanone monooxygenase derived from the Rhodococcus sp. Phil is a mutant with a sequence as shown in SEQ ID NO: 11 or SEQ ID NO: 12; and the cyclohexanone monooxygenase derived from the Rhodococcus ruber-SDl is a mutant with a sequence as shown in SEQ ID NO: 14 or SEQ ID NO: 15;
preferably, the ammonia lyase is derived from photorhabdus luminescens or Solenostemon scutellarioides;
preferably, the ene reductase is derived from Saccharomyces cerevisiae or Chryseobacterium sp. CA49;
preferably, the imine reductase is derived from Streptomyces sp or Bacillus cereus;
preferably, the amino acid dehydrogenase is a leucine dehydrogenase derived from Bacillus cereus or a phenylalanine dehydrogenase derived from Bacillus sphaericus; and
preferably, the nitrilase is derived from Aspergillus niger CBS 513.88 or Neurospora crassa OR74A .

3. The immobilized enzyme as claimed in claim 1, wherein the amino resin carrier is an amino resin carrier with a C2 or C4 linker arm.

4. The immobilized enzyme as claimed in claim 1, wherein the cross-linking agent is a glutaraldehyde, and the macromolecular polymer is a PEG or a PEI.

5. A preparation method for an immobilized enzyme, comprising:
performing pretreatment on a cross-linking agent with a macromolecular polymer, to obtain a modified cross-linking agent;
performing modification on an amino resin carrier with the treated cross-linking agent, to obtain a modified carrier; and
immobilizing an enzyme to on the modified carrier, to obtain the immobilized enzyme;
wherein the enzyme is selected from any one of the group consisting of a transaminase, a ketoreductase, a monooxygenase, an ammonia lyase, an ene reductase, an imine reductase, an amino acid dehydrogenase and a nitrilase.

6. The preparation method as claimed in claim 5, wherein the transaminase is derived from Chromobacterium violaceum DSM3019, Aspergillus fumigatus, or Arthrobacter citreus, preferably, the transaminase derived from the Chromobacterium violaceum DSM30191 is a mutant with a sequence as shown in SEQ ID NO: 2 or SEQ ID NO: 3; and the transaminase derived from the Arthrobacter citreus is a mutant with a sequence as shown in SEQ ID NO: 5 or SEQ ID NO: 6;
preferably, the ketoreductase is derived from Acetobacter sp. CCTCC M209061 or Candida macedoniensis AKU4588, more preferably the ketoreductase derived from the Acetobacter sp. CCTCC M209061 is a mutant with a sequence as shown in SEQ ID NO: 8 or SEQ ID NO: 9;
preferably, the monooxygenase is a cyclohexanone monooxygenase derived from Rhodococcus sp. Phil, or a cyclohexanone monooxygenase derived from Brachymonas petroleovorans, or a monooxygenase derived from Rhodococcus ruber-SD1 more preferably, the cyclohexanone monooxygenase derived from the Rhodococcus sp. Phil is a mutant with a sequence as shown in SEQ ID NO: 11 or SEQ ID NO: 12; and the cyclohexanone monooxygenase derived from the Rhodococcus ruber-SD1 is a mutant with a sequence as shown in SEQ ID NO: 14 or SEQ ID NO: 15;
preferably, the ammonia lyase is derived from photorhabdus luminescens or Solenostemon scutellarioides;
preferably, the ene reductase is derived from Saccharomyces cerevisiae or Chryseobacterium sp. CA49;
preferably, the imine reductase is derived from Streptomyces sp or Bacillus cereus;
preferably, the amino acid dehydrogenase is a leucine dehydrogenase derived from Bacillus cereus or a phenylalanine dehydrogenase derived from Bacillus sphaericus; and
preferably, the nitrilase is derived from Aspergillus niger CBS 513.88 or Neurospora crassa OR74A .

7. The preparation method as claimed in claim 5, wherein the amino resin carrier is an amino resin carrier with a C2 or C4 linker arm.

8. The preparation method as claimed in claim 5, wherein the cross-linking agent is a glutaraldehyde, and the macromolecular polymer is a PEG or a PEI.

9. The preparation method as claimed in claim 8, wherein a mass ratio of the PEG and the glutaraldehyde is 1:1-10:1, preferably 2:1-5:1.

10. (canceled)

11. (canceled)

12. The immobilized enzyme as claimed in claim 1, wherein the amino resin carrier is selected from any one of the group consisting of: SEPLITE® LX1000EA, LX1000HA, LX1000NH, LX1000EPN, HM100D, Purolite® Lifetech™ ECR8309, ECR8409, ECR8305, ECR8404, ECR8315, ECR8415, HECHENG® ESR-1, ESR-3, ESR-5 and ESR-8.

13. The immobilized enzyme as claimed in claim 4, wherein the PEG is selected from any one of PEG400-PEG6000.

14. The immobilized enzyme as claimed in claim 4, wherein the PEI is selected from a PEI with 3-70 KDa of a molecular weight.

15. The immobilized enzyme as claimed in claim 4, wherein a mass ratio of the PEG and the glutaraldehyde is 1:1-10:1, further preferably 2:1-5:1.

16. The immobilized enzyme as claimed in claim 4, wherein a mass ratio of the PEI and the glutaraldehyde is 3:1-1:5.

17. The immobilized enzyme as claimed in claim 16, wherein the mass ratio of the PEI and the glutaraldehyde is 1:1-1:2.

18. The preparation method as claimed in claim 7, wherein the amino resin carrier is selected from any one of the group consisting of: SEPLITE® LX1000EA, LX1000HA, LX1000NH, LX1000EPN, HM100D, Purolite® Lifetech™ ECR8309, ECR8409, ECR8305, ECR8404, ECR8315, ECR8415, HECHENG® ESR-1, ESR-3, ESR-5 and ESR-8.

19. The preparation method as claimed in claim 8, wherein the PEG is selected from any one of PEG400-PEG6000.

20. The preparation method as claimed in claim 8, wherein the PEI is selected from a PEI with 3-70 KDa of a molecular weight.

21. The preparation method as claimed in claim 9, wherein a mass ratio of the PEI and the glutaraldehyde is 3:1 -1:5.

22. The preparation method as claimed in claim 21, wherein a mass ratio of the PEI and the glutaraldehyde is 1:1-1:2.


SEQUENCE LISTING (15 sequences)

SEQ ID NO: 1 - protein, 459 aa, Chromobacterium violaceum
  SITE (1)..(459): TA-Cv-mather line
MQKQRTTSQWRELDAAHHLHPFTDTASLNQAGARVMTRGEGVYLWDSEGNKIIDGMAGLWCVNVGYGRKDFAEAARRQMEELPFYNTFFKTTHPAVVELSSLLAEVTPAGFDRVFYTNSGSESVDTMIRMVRRYWDVQGKPEKKTLIGRWNGYHGSTIGGASLGGMKYMHEQGDLPIPGMAHIEQPWWYKHGKDMTPDEFGVVAARWLEEKILEIGADKVAAFVGEPIQGAGGVIVPPATYWPEIERICRKYDVLLVADEVICGFGRTGEWFGHQHFGFQPDLFTAAKGLSSGYLPIGAVFVGKRVAEGLIAGGDFNHGFTYSGHPVCAAVAHANVAALRDEGIVQRVKDDIGPYMQKRWRETFSRFEHVDDVRGVGMVQAFTLVKNKAKRELFPDFGEIGTLCRDIFFRNNLIMRACGDHIVSAPPLVMTRAEVDEMLAVAERCLEEFEQTLKARGLA

SEQ ID NO: 2 - protein, 459 aa, Chromobacterium violaceum
  VARIANT (1)..(459): TA-Cv-V1
  MUTAGEN (1)..(459): R416T+T7C+S47C+Q380L
MQKQRTCSQWRELDAAHHLHPFTDTASLNQAGARVMTRGEGVYLWDCEGNKIIDGMAGLWCVNVGYGRKDFAEAARRQMEELPFYNTFFKTTHPAVVELSSLLAEVTPAGFDRVFYTNSGSESVDTMIRMVRRYWDVQGKPEKKTLIGRWNGYHGSTIGGASLGGMKYMHEQGDLPIPGMAHIEQPWWYKHGKDMTPDEFGVVAARWLEEKILEIGADKVAAFVGEPIQGAGGVIVPPATYWPEIERICRKYDVLLVADEVICGFGRTGEWFGHQHFGFQPDLFTAAKGLSSGYLPIGAVFVGKRVAEGLIAGGDFNHGFTYSGHPVCAAVAHANVAALRDEGIVQRVKDDIGPYMQKRWRETFSRFEHVDDVRGVGMVLAFTLVKNKAKRELFPDFGEIGTLCRDIFFRNNLIMTACGDHIVSAPPLVMTRAEVDEMLAVAERCLEEFEQTLKARGLA

SEQ ID NO: 3 - protein, 459 aa, Chromobacterium violaceum
  VARIANT (1)..(459): TA-Cv-V2
  MUTAGEN (1)..(459): R416T+T7C+S47C+R405E+K90G+A95P+K304D+Q380L+I297L
MQKQRTCSQWRELDAAHHLHPFTDTASLNQAGARVMTRGEGVYLWDCEGNKIIDGMAGLWCVNVGYGRKDFAEAARRQMEELPFYNTFFGTTHPPVVELSSLLAEVTPAGFDRVFYTNSGSESVDTMIRMVRRYWDVQGKPEKKTLIGRWNGYHGSTIGGASLGGMKYMHEQGDLPIPGMAHIEQPWWYKHGKDMTPDEFGVVAARWLEEKILEIGADKVAAFVGEPIQGAGGVIVPPATYWPEIERICRKYDVLLVADEVICGFGRTGEWFGHQHFGFQPDLFTAAKGLSSGYLPLGAVFVGDRVAEGLIAGGDFNHGFTYSGHPVCAAVAHANVAALRDEGIVQRVKDDIGPYMQKRWRETFSRFEHVDDVRGVGMVLAFTLVKNKAKRELFPDFGEIGTLCEDIFFRNNLIMTACGDHIVSAPPLVMTRAEVDEMLAVAERCLEEFEQTLKARGLA

SEQ ID NO: 4 - protein, 476 aa, Arthrobacter citreus
  SITE (1)..(476): TA-Ac-mather line
MGLTVQKINWEQVKEWDRKYLMRTFSTQNEYQPVPIESTEGDYLITPGGTRLLDFFNQLCCVNLGQKNQKVNAAIKEALDRYGFVWDTYATDYKAKAAKIIIEDILGDEDWPGKVRFVSTGSEAVETALNIARLYTNRPLVVTREHDYHGWTGGAATVTRLRSFRSGLVGENSESFSAQIPGSSCSSAVLMAPSSNTFQDSNGNYLKDENGELLSVKYTRRMIENYGPEQVAAVITEVSQGVGSTMPPYEYVPQIRKMTKELGVLWISDEVLTGFGRTGKWFGYQHYGVQPDIITMGKGLSSSSLPAGAVVVSKEIAAFMDKHRWESVSTYAGHPVAMAAVCANLEVMMEENLVEQAKNSGEYIRSKLELLQEKHKSIGNFDGYGLLWIVDIVNAKTKTPYVKLDRNFRHGMNPNQIPTQIIMEKALEKGVLIGGAMPNTMRIGASLNVSRGDIDKAMDALDYALDYLESGEWQQS

SEQ ID NO: 5 - protein, 476 aa, Arthrobacter citreus
  VARIANT (1)..(476): TA-Ac -V1
  MUTAGEN (1)..(476): L3S+V5S+C60Y+F164L+A178L+S187A+I180V+L370A+G411D+S186G+Y384F+I389 F+V252I+L404Q+E171D
MGSTSQKINWEQVKEWDRKYLMRTFSTQNEYQPVPIESTEGDYLITPGGTRLLDFFNQLYCVNLGQKNQKVNAAIKEALDRYGFVWDTYATDYKAKAAKIIIEDILGDEDWPGKVRFVSTGSEAVETALNIARLYTNRPLVVTREHDYHGWTGGAATVTRLRSLRSGLVGDNSESFSLQVPGSSCGAAVLMAPSSNTFQDSNGNYLKDENGELLSVKYTRRMIENYGPEQVAAVITEVSQGVGSTMPPYEYIPQIRKMTKELGVLWISDEVLTGFGRTGKWFGYQHYGVQPDIITMGKGLSSSSLPAGAVVVSKEIAAFMDKHRWESVSTYAGHPVAMAAVCANLEVMMEENLVEQAKNSGEYIRSKLEALQEKHKSIGNFDGFGLLWFVDIVNAKTKTPYVKQDRNFRHDMNPNQIPTQIIMEKALEKGVLIGGAMPNTMRIGASLNVSRGDIDKAMDALDYALDYLESGEWQQS

SEQ ID NO: 6 - protein, 476 aa, Arthrobacter citreus
  VARIANT (1)..(476): TA-Ac -V2
  MUTAGEN (1)..(476): L3S+V5S+C60Y+F164L+A178L+S187A+I180V+L370A+G411D+S186G+Y384F+I389 F+V252I+E424Q+M423K
MGSTSQKINWEQVKEWDRKYLMRTFSTQNEYQPVPIESTEGDYLITPGGTRLLDFFNQLYCVNLGQKNQKVNAAIKEALDRYGFVWDTYATDYKAKAAKIIIEDILGDEDWPGKVRFVSTGSEAVETALNIARLYTNRPLVVTREHDYHGWTGGAATVTRLRSLRSGLVGENSESFSLQVPGSSCGAAVLMAPSSNTFQDSNGNYLKDENGELLSVKYTRRMIENYGPEQVAAVITEVSQGVGSTMPPYEYIPQIRKMTKELGVLWISDEVLTGFGRTGKWFGYQHYGVQPDIITMGKGLSSSSLPAGAVVVSKEIAAFMDKHRWESVSTYAGHPVAMAAVCANLEVMMEENLVEQAKNSGEYIRSKLEALQEKHKSIGNFDGFGLLWFVDIVNAKTKTPYVKLDRNFRHDMNPNQIPTQIIKQKALEKGVLIGGAMPNTMRIGASLNVSRGDIDKAMDALDYALDYLESGEWQQS

SEQ ID NO: 7 - protein, 253 aa, Acetobacter sp.
  SITE (1)..(253): KRED-Ac-mather line
MARVAGKVAIVSGAANGIGKATAQLLAKEGAKVVIGDLKEEDGQKAVAEIKAAGGEAAFVKLNVTDEAAWKAAIGQTLKLYGRLDIAVNNAGIAYSGSVESTSLEDWRRVQSINLDGVFLGTQVAIEAMKKSGGGSIVNLSSIEGLIGDPMLAAYNASKGGVRLFTKSAALHCAKSGYKIRVNSVHPGYIWTPMVAGLTKEDAAARQKLVDLHPIGHLGEPNDIAYGILYLASDESKFVTGSELVIDGGYTAQ

SEQ ID NO: 8 - protein, 253 aa, Acetobacter sp.
  VARIANT (1)..(253): KRED-Ac-V1
  MUTAGEN (1)..(253): E144S+A94N+N156V
MARVAGKVAIVSGAANGIGKATAQLLAKEGAKVVIGDLKEEDGQKAVAEIKAAGGEAAFVKLNVTDEAAWKAAIGQTLKLYGRLDIAVNNAGINYSGSVESTSLEDWRRVQSINLDGVFLGTQVAIEAMKKSGGGSIVNLSSISGLIGDPMLAAYVASKGGVRLFTKSAALHCAKSGYKIRVNSVHPGYIWTPMVAGLTKEDAAARQKLVDLHPIGHLGEPNDIAYGILYLASDESKFVTGSELVIDGGYTAQ

SEQ ID NO: 9 - protein, 253 aa, Acetobacter sp.
  VARIANT (1)..(253): KRED-Ac-V2
  MUTAGEN (1)..(253): E144S+A94T+N156T
MARVAGKVAIVSGAANGIGKATAQLLAKEGAKVVIGDLKEEDGQKAVAEIKAAGGEAAFVKLNVTDEAAWKAAIGQTLKLYGRLDIAVNNAGITYSGSVESTSLEDWRRVQSINLDGVFLGTQVAIEAMKKSGGGSIVNLSSISGLIGDPMLAAYTASKGGVRLFTKSAALHCAKSGYKIRVNSVHPGYIWTPMVAGLTKEDAAARQKLVDLHPIGHLGEPNDIAYGILYLASDESKFVTGSELVIDGGYTAQ

SEQ ID NO: 10 - protein, 541 aa, Rhodococcus sp. Phi1
  SITE (1)..(541): CHMO-Rs-mather line
MTAQISPTVVDAVVIGAGFGGIYAVHKLHNEQGLTVVGFDKADGPGGTWYWNRYPGALSDTESHLYRFSFDRDLLQDGTWKTTYITQPEILEYLESVVDRFDLRRHFRFGTEVTSAIYLEDENLWEVSTDKGEVYRAKYVVNAVGLLSAINFPDLPGLDTFEGETIHTAAWPEGKNLAGKRVGVIGTGSTGQQVITALAPEVEHLTVFVRTPQYSVPVGNRPVTKEQIDAIKADYDGIWDSVKKSAVAFGFEESTLPAMSVSEEERNRIFQEAWDHGGGFRFMFGTFGDIATDEAANEAAASFIRSKIAEIIEDPETARKLMPTGLYAKRPLCDNGYYEVYNRPNVEAVAIKENPIREVTAKGVVTEDGVLHELDVLVFATGFDAVDGNYRRIEIRGRNGLHINDHWDGQPTSYLGVTTANFPNWFMVLGPNGPFTNLPPSIETQVEWISDTVAYAERNEIRAIEPTPEAEEEWTQTCTDIANATLFTRGDSWIFGANVPGKKPSVLFYLGGLGNYRNVLAGVVADSYRGFELKSAVPVTA

SEQ ID NO: 11 - protein, 541 aa, Rhodococcus sp. Phi1
  VARIANT (1)..(541): CHMO-Rs-V1
  MUTAGEN (1)..(541): F508Y+F435N+L438A+T436S+F280V+S441V
MTAQISPTVVDAVVIGAGFGGIYAVHKLHNEQGLTVVGFDKADGPGGTWYWNRYPGALSDTESHLYRFSFDRDLLQDGTWKTTYITQPEILEYLESVVDRFDLRRHFRFGTEVTSAIYLEDENLWEVSTDKGEVYRAKYVVNAVGLLSAINFPDLPGLDTFEGETIHTAAWPEGKNLAGKRVGVIGTGSTGQQVITALAPEVEHLTVFVRTPQYSVPVGNRPVTKEQIDAIKADYDGIWDSVKKSAVAFGFEESTLPAMSVSEEERNRIFQEAWDHGGGVRFMFGTFGDIATDEAANEAAASFIRSKIAEIIEDPETARKLMPTGLYAKRPLCDNGYYEVYNRPNVEAVAIKENPIREVTAKGVVTEDGVLHELDVLVFATGFDAVDGNYRRIEIRGRNGLHINDHWDGQPTSYLGVTTANFPNWFMVLGPNGPNSNAPPVIETQVEWISDTVAYAERNEIRAIEPTPEAEEEWTQTCTDIANATLFTRGDSWIFGANVPGKKPSVLYYLGGLGNYRNVLAGVVADSYRGFELKSAVPVTA

SEQ ID NO: 12 - protein, 541 aa, Rhodococcus sp. Phi1
  VARIANT (1)..(541): CHMO-Rs-V2
  MUTAGEN (1)..(541): F508Y+F435N+L438A+T436S+F280V+S441V+L510V
MTAQISPTVVDAVVIGAGFGGIYAVHKLHNEQGLTVVGFDKADGPGGTWYWNRYPGALSDTESHLYRFSFDRDLLQDGTWKTTYITQPEILEYLESVVDRFDLRRHFRFGTEVTSAIYLEDENLWEVSTDKGEVYRAKYVVNAVGLLSAINFPDLPGLDTFEGETIHTAAWPEGKNLAGKRVGVIGTGSTGQQVITALAPEVEHLTVFVRTPQYSVPVGNRPVTKEQIDAIKADYDGIWDSVKKSAVAFGFEESTLPAMSVSEEERNRIFQEAWDHGGGVRFMFGTFGDIATDEAANEAAASFIRSKIAEIIEDPETARKLMPTGLYAKRPLCDNGYYEVYNRPNVEAVAIKENPIREVTAKGVVTEDGVLHELDVLVFATGFDAVDGNYRRIEIRGRNGLHINDHWDGQPTSYLGVTTANFPNWFMVLGPNGPNSNAPPVIETQVEWISDTVAYAERNEIRAIEPTPEAEEEWTQTCTDIANATLFTRGDSWIFGANVPGKKPSVLYYVGGLGNYRNVLAGVVADSYRGFELKSAVPVTA

SEQ ID NO: 13 - protein, 603 aa, Rhodococcus ruber-SD1
  SITE (1)..(603): CHMO-Rr-mather line
MTTSIDREALRRKYAEERDKRIRPDGNDQYIRLDHVDGWSHDPYMPITPREPKLDHVTFAFIGGGFSGLVTAARLRESGVESVRIIDKAGDFGGVWYWNRYPGAMCDTAAMVYMPLLEETGYMPTEKYAHGPEILEHCQRIGKHYDLYDDALFHTEVTDLVWQEHDQRWRISTNRGDHFTAQFVGMGTGPLHVAQLPGIPGIESFRGKSFHTSRWDYDYTGGDALGAPMDKLADKRVAVIGTGATAVQCVPELAKYCRELYVVQRTPSAVDERGNHPIDEKWFAQIATPGWQKRWLDSFTAIWDGVLTDPSELAIEHEDLVQDGWTALGQRMRAAVGSVPIEQYSPENVQRALEEADDEQMERIRARVDEIVTDPATAAQLKAWFRQMCKRPCFHDDYLPAFNRPNTHLVDTGGKGVERITENGVVVAGVEYEVDCIVYASGFEFLGTGYTDRAGFDPTGRDGVKLSEHWAQGTRTLHGMHTYGFPNLFVLQLMQGAALGSNIPHNFVEAARVVAAIVDHVLSTGTSSVETTKEAEQAWVQLLLDHGRPLGNPECTPGYYNNEGKPAELKDRLNVGYPAGSAAFFRMMDHWLAAGSFDGLTFR

SEQ ID NO: 14 - protein, 603 aa, Rhodococcus ruber-SD1
  VARIANT (1)..(603): CHMO-Rr-V1
  MUTAGEN (1)..(603): P190L+Y559M+C249V+C393V+C257A+M45T
MTTSIDREALRRKYAEERDKRIRPDGNDQYIRLDHVDGWSHDPYTPITPREPKLDHVTFAFIGGGFSGLVTAARLRESGVESVRIIDKAGDFGGVWYWNRYPGAMCDTAAMVYMPLLEETGYMPTEKYAHGPEILEHCQRIGKHYDLYDDALFHTEVTDLVWQEHDQRWRISTNRGDHFTAQFVGMGTGLLHVAQLPGIPGIESFRGKSFHTSRWDYDYTGGDALGAPMDKLADKRVAVIGTGATAVQVVPELAKYARELYVVQRTPSAVDERGNHPIDEKWFAQIATPGWQKRWLDSFTAIWDGVLTDPSELAIEHEDLVQDGWTALGQRMRAAVGSVPIEQYSPENVQRALEEADDEQMERIRARVDEIVTDPATAAQLKAWFRQMCKRPVFHDDYLPAFNRPNTHLVDTGGKGVERITENGVVVAGVEYEVDCIVYASGFEFLGTGYTDRAGFDPTGRDGVKLSEHWAQGTRTLHGMHTYGFPNLFVLQLMQGAALGSNIPHNFVEAARVVAAIVDHVLSTGTSSVETTKEAEQAWVQLLLDHGRPLGNPECTPGMYNNEGKPAELKDRLNVGYPAGSAAFFRMMDHWLAAGSFDGLTFR

SEQ ID NO: 15 - protein, 603 aa, Rhodococcus ruber-SD1
  VARIANT (1)..(603): CHMO-Rr-V2
  MUTAGEN (1)..(603): Y559M+P190L+P504V
MTTSIDREALRRKYAEERDKRIRPDGNDQYIRLDHVDGWSHDPYMPITPREPKLDHVTFAFIGGGFSGLVTAARLRESGVESVRIIDKAGDFGGVWYWNRYPGAMCDTAAMVYMPLLEETGYMPTEKYAHGPEILEHCQRIGKHYDLYDDALFHTEVTDLVWQEHDQRWRISTNRGDHFTAQFVGMGTGLLHVAQLPGIPGIESFRGKSFHTSRWDYDYTGGDALGAPMDKLADKRVAVIGTGATAVQCVPELAKYCRELYVVQRTPSAVDERGNHPIDEKWFAQIATPGWQKRWLDSFTAIWDGVLTDPSELAIEHEDLVQDGWTALGQRMRAAVGSVPIEQYSPENVQRALEEADDEQMERIRARVDEIVTDPATAAQLKAWFRQMCKRPCFHDDYLPAFNRPNTHLVDTGGKGVERITENGVVVAGVEYEVDCIVYASGFEFLGTGYTDRAGFDPTGRDGVKLSEHWAQGTRTLHGMHTYGFPNLFVLQLMQGAALGSNIVHNFVEAARVVAAIVDHVLSTGTSSVETTKEAEQAWVQLLLDHGRPLGNPECTPGMYNNEGKPAELKDRLNVGYPAGSAAFFRMMDHWLAAGSFDGLTFR